(19) United States
(12) Patent Application Publication
(10) Pub. No.: US 2010/0233252 A1
(43) Pub. Date: Sep. 16, 2010

(54) Low-Moisture-Content Hard Capsule and Production Method Therefor

(75) Inventors: Shinji Tochio, Yamatokoriyama-shi (JP); Julio A. Prado, Alcobendas (ES)

Correspondence Address: FITCH, EVEN, TABIN & FLANNERY, P. O. BOX 18415, WASHINGTON, DC, 20036, US

(73) Assignee: QUALICAPS CO., LTD., Yamatokoriyama-shi (JP)

(21) Appl. No.: 12/223,117

(22) PCT Filed: Jan. 24, 2007
(86) PCT No.: PCT/JP2007/051527

§ 371 Date: Jul. 23, 2008

Related U.S. Application Data
(60) Provisional application No. 60/761,345, filed on Jan. 24, 2006.

(51) Int. Cl. A61K 9/48; A23L 1/00; A61K 8/11; B29C 39/00

(52) U.S. Cl. 424/451; 426/89; 424/401; 264/319

(57) ABSTRACT

A method is provided for the production of hard capsules having a low moisture content and low hygroscopicity, as well as a method for reducing the moisture content and hygroscopicity of hard capsules produced by a gel cooling process using, as principal components, a water-soluble cellulose compound and a gelling agent. The hard capsules have a water-soluble cellulose compound, gelling agent, and, if necessary, a gelling aid that has loss on drying, after 10 days of storage at 25° C. and a relative humidity of 53%, of less than 6% by weight. Such a hard capsule can be produced by heating a gelled capsule film to 50-150° C. before, after, or simultaneously with a drying step after a gelling step in a gel-cooling process for producing a hard capsule having a water-soluble cellulose compound and a gelling agent as principal components.

### TECHNICAL FIELD

The present invention relates to a hard capsule having a low moisture content and a low hygroscopicity, and a method for producing the same. More specifically, the present invention relates to a hard capsule produced using a water-soluble cellulose compound and a gelling agent as principal components according to a gel cooling process, and having lower moisture content and a lower hygroscopicity than hitherto known hard capsules, and a method for producing such a hard capsule. The gel cooling process is a method for producing a capsule, which utilizes the property that a mixture of the water-soluble cellulose compound and the gelling agent forms gel at temperatures of 50° C. or lower.

The invention also relates to a method for reducing the moisture content and a hygroscopicity of a hard capsule produced by a gel cooling process using a water-soluble cellulose compound and a gelling agent as principal components.

### BACKGROUND OF THE INVENTION

Heretofore, gelatin capsules have mainly been used as hard capsules for applications with drugs, quasi drugs, food products, etc. However, the gelatin capsule has a disadvantage in that when the moisture content of the capsule film is reduced to 11% or less, its strength sharply decreases. For example, when a hygroscopic substance is inserted into the gelatin capsule and the hygroscopic substance absorbs the moisture in the capsule film, the gelatin capsule becomes weak and is likely to break. For example, low-molecular-weight polyethylene glycols such as polyethylene glycols 200 to 600, glycerine fatty acid esters, and medium-chain-fatty acid triglycerides are widely used as excipients due to their outstanding solubilities and absorbabilities. However, these substances are hygroscopic, and therefore are difficult to prepare capsule products, into which drugs, food products, or cosmetic materials are inserted, using a gelatin capsule without compromised strength reduction. Since the moisture content of the gelatin capsule cannot be reduced for the above-described reasons, and is relatively as high as 15%, the gelatin capsule cannot be used to contain substances that exhibit moisture reactivity or substances that are unstable in the presence of moisture (Patent Document 1, etc.).

Therefore, as non-gelatin hard capsules that are free from the above-described disadvantages of the gelatin capsule, capsules comprising a water-soluble cellulose compound as a capsule-preparing agent and capsules in which polyvinyl alcohol and/or a gelling agent are combined with the water-soluble cellulose compound are proposed (Patent Documents 1 to 4, etc.). In particular, capsules comprising hydroxypropyl methylcellulose (HPMC) as a water-soluble cellulose compound (HPMC capsule) exhibit good strength under low-moisture conditions, and are excellent in that they can contain substances that are highly hygroscopic and/or highly reactive to moisture.

Although the moisture content of the film of such HPMC capsules is markedly lower than that of the film of gelatin capsules, the moisture content is still high enough to affect substances that are highly hygroscopic and/or highly reactive to moisture. Thus, further improvement is required to provide capsule products with higher stability.

Patent Document 1: Japanese Unexamined Patent Publication No. 1991-279325

Patent Document 2: Japanese Examined Patent Publication No. 1972-4310

Patent Document 3: Japanese Unexamined Patent Publication No. 1986-100519

Patent Document 4: Japanese Unexamined Patent Publication No. 1986-266060

### BRIEF DESCRIPTION OF THE DRAWINGS

FIG. 1 shows graphs of the relationships between heating temperature (from 50 to 150° C.) used in capsule formation and capsule-film moisture content (loss on drying)(%).

FIG. 2 shows moisture adsorption/desorption isotherms of moisture adsorption/desorption to/from the capsule film obtained in Experimental Example 2 for the gel-cooling type hard capsules according to Formulation 11 prepared by heating at 70° C. for 1 hr and for the gel-cooling type hard capsules as a control sample prepared without heating.

FIG. 3 shows moisture adsorption/desorption isotherms of moisture adsorption/desorption to/from the capsule film obtained in Experimental Example 3 for the gel-cooling type hard capsules according to Formulations 10 and 11 prepared by heating for 1 hr at 50 and 70° C., respectively, and for the gel-cooling type hard capsules as a control sample prepared without heating.

FIG. 4 shows only the moisture adsorption isotherms taken from FIG. 3.

FIG. 5 shows graphs of the relationship between moisture content in capsule film and shock resistance obtained in Experimental Example 4 for each type of capsules.

### DISCLOSURE OF THE INVENTION

The invention was accomplished in view of the above-described problems, and aims to provide hard capsules that have a low moisture content, particularly a low moisture content and low hygroscopicity, while maintaining film strength, and that can be suitably used to contain substances that are highly reactive to moisture, substances with high hygroscopicity, or substances with a high moisture content. The invention also aims to provide a method for producing such a hard capsule. In addition, the invention aims to provide a method for reducing the moisture content and hygroscopicity of hard capsules produced by a gel cooling process using a water-soluble cellulose compound and a gelling agent as principal components.

In order to achieve the above-described objects, the inventors conducted extensive research day and night, and found that it is possible to provide a capsule, whose moisture content is lower than that of not only gelatin capsules but also hitherto-known hard capsules produced using a water-soluble cellulose compound and a gelling agent as principal components by a gel cooling process (hereinafter sometimes referred to as “cold-gel hard capsule”), by adding the steps of heating the gelled capsule film at 50° C. or higher to hitherto-known process for producing the cold-gel hard capsule. The cold-gel hard capsule of the present invention obtained by the above-described method has lower hygroscopicity, and is unlikely to be affected by external humidity as compared with hitherto-known cold-gel hard capsules. It was confirmed that the cold-gel hard capsule of the present invention can be suitably used to contain substances that are highly reactive to moisture or that are high in moisture content or hygroscopicity.

The present invention was accomplished based on these findings, and encompasses the following aspects.

Item 1. A hard capsule comprising a water-soluble cellulose compound, a gelling agent, and, if required, a gelling aid;

the hard capsule having loss on drying, after 10 days of storage at 25° C. and at a relative humidity of 53%, of less than 6% by weight.

Item 2. The hard capsule according to item 1, wherein loss on drying, after 10 days of storage at 25° C. and at a relative humidity of 12%, 22%, 33%, or 43% is 1.1% by weight or less, 2.1% by weight or less, 3.2% by weight or less, or 4.7% by weight or less, respectively.
Item 3. The hard capsule according to item 1, wherein hygroscopicity (%) of a capsule film under conditions of a temperature of 25° C. and each relative humidity value shown below satisfies at least one of the following conditions (1) to (5):

(1) 9.2% or less at a relative humidity of 12%;

(2) 9.5% or less at a relative humidity of 22%;

(3) 9.7% or less at a relative humidity of 33%;

(4) 10.9% or less at a relative humidity of 43%;

(5) 11.1% or less at a relative humidity of 53%.

Item 4. The hard capsule according to item 1, wherein the water-soluble cellulose compound is cellulose ether substituted with at least one group selected from C₁₋₆ alkyl groups and C₁₋₆ hydroxyalkyl groups.
Item 5. The hard capsule according to item 3, wherein the water-soluble cellulose compound is hydroxypropyl methylcellulose.
Item 6. The hard capsule according to item 1, wherein the gelling agent is at least one member selected from the group consisting of carrageenan, pectin, xanthan gum, locust bean gum, tamarind seed polysaccharide, curdlan, gelatin, fur selenium, agar, and gellan gum.
Item 7. The hard capsule according to item 5, wherein the gelling agent is carrageenan.
Item 8. The hard capsule according to item 1, wherein the water-soluble cellulose compound is hydroxypropyl methylcellulose, the gelling agent is carrageenan, and the gelling aid is potassium chloride.
Item 9. The hard capsule according to item 7, comprising 70 to 99.9% by weight of hydroxypropyl methylcellulose as the water-soluble cellulose compound, 0.05 to 2.2% by weight of carrageenan as the gelling agent, and 0.05 to 2.2% by weight of potassium chloride as the gelling aid.
Item 10. The hard capsule according to item 1 prepared by the following steps:

(1) a step of dipping a capsule-mold pin into a capsule-preparing solution comprising a water-soluble cellulose compound and a gelling agent, and, if required, a gelling aid;

(2) a step of drawing the capsule-mold pin out of the capsule-preparing solution, and gelling the solution adhering to an outside surface of the capsule-mold pin at a temperature of 35° C. or less;

(3) a step of drying the gelled capsule film formed covering the outside surface of the capsule-mold pin;

(4) a step of removing the dried capsule film from the capsule-mold pin, and

(5) a step of heating the gelled and/or dried capsule film to 50 to 150° C.; after step (2); before, after, or simultaneously with step (3); or after step (4).

Item 11. The hard capsule according to item 9, which is prepared by performing the heating step before, after, or simultaneously with step (3) of drying the gelled capsule film formed covering the outside surface of the capsule-mold pin.
Item 12. A capsule product in which an ingredient is inserted into a hard capsule of any one of items 1 to 10.
Item 13. The capsule product according to item 11, in which the ingredient is a drug, food, or cosmetic material.
Item 14. A method for producing a hard capsule comprising:

(1) dipping a capsule-mold pin into a capsule-preparing solution comprising a water-soluble cellulose compound and a gelling agent, and, if required, a gelling aid;

(2) drawing the capsule-mold pin out of the capsule-preparing solution, and gelling the solution adhering to an outside surface of the capsule-mold pin at a temperature of 35° C. or less;

(3) drying the gelled capsule film formed covering the outside surface of the capsule-mold pin;

(4) removing the dried capsule film from the capsule-mold pin; and

(5) heating the gelled and/or dried capsule film to 50 to 150° C.; after step (2); before, after, or simultaneously with step (3); or after step (4).

Item 15. The method for producing a hard capsule according to item 13, comprising the heating step before, after, or simultaneously with step (3) of drying the gelled capsule film formed covering the outside surface of the capsule-mold pin.
Item 16. The method for producing a hard capsule according to item 13, the hard capsule having loss on drying, after 10 days of storage at 25° C. and at a relative humidity of 53%, of less than 6% by weight.
Item 17. A method for reducing a moisture content and hygroscopicity of a hard capsule comprising as a main component a water-soluble cellulose compound, a gelling agent, and, if required, a gelling aid, the method comprising:

(a) dipping a capsule-mold pin into a capsule-preparing solution comprising a water-soluble cellulose compound and a gelling agent, and, if required, a gelling aid;

(b) drawing the capsule-mold pin out of the capsule-preparing solution, and gelling the solution adhering to an outside surface of the capsule-mold pin at a temperature of 35° C. or less;

(c) drying the gelled capsule film formed covering the outside surface of the capsule-mold pin; and

(d) removing the dried capsule film from the capsule-mold pin,

wherein the gelled and/or dried capsule film is heated to 50 to 150° C.; after step (b); before, after, or simultaneously with step (c); or after step (d).

### BEST MODE FOR CARRYING OUT THE INVENTION

### I. A Low-Moisture Hard Capsule and a Method for Producing the Same

The hard capsule of the invention comprises a water-soluble cellulose compound and a gelling agent as principal components.

Cellulose ethers substituted with at least one group of alkyl groups and hydroxyalkyl groups can be mentioned as usable water-soluble cellulose compounds for the invention. The “alkyl group” in the above-mentioned alkyl groups and hydroxyalkyl groups refers to linear or branched lower alkyl groups having 1 to 6 carbon atoms, and preferably 1 to 4 carbon atoms; and a methyl group, an ethyl group, a butyl group, and a propyl group can be specifically mentioned. Specific examples of water-soluble cellulose compounds include lower alkylcelluloses, such as methylcellulose and the like; lower hydroxyalkyl celluloses, such as hydroxyethylcellulose, hydroxypropylcellulose, and the like; and lower hydroxyalkyl alkylcelluloses, such as hydroxyethyl methylcellulose, hydroxyethyl ethylcellulose, hydroxypropyl methylcellulose, and the like. Hydroxypropyl methylcellulose is particularly excellent with regard to film formability and mechanical strength under low moisture conditions, and thus is an optimal water-soluble cellulose compound.

Carrageenan, tamarind seed polysaccharide, pectin, xanthan gum, locust bean gum, curdlan, gelatin, fur selenium, agar, gellan gum, etc. can be mentioned as usable gelling agents for the invention. They can be used alone or in combination.

Among the above-mentioned gelling agents, carrageenan has a high gel strength and exhibits an excellent gelling ability when used in a small amount and in combination with specific ions, and thus is an optimal gelling agent.

In general, three kinds of carrageenan, that is, κ-carrageenan, ι-carrageenan, and λ-carrageenan, are known. In the invention, κ-carrageenan and ι-carrageenan, which have a gelling ability, can be suitably used. Pectins can be classified into LM pectins and HM pectins according to the difference in the degree of esterification. Gellan gum can also be classified into acylation gellan gum (native gellan gum) and deacylation gellan gum according to the presence or absence of acylation. In the invention, any pectin can be used without distinguishing the degree of esterification, and any gellan gum can be used without distinguishing the existence of acylation.

For the hard capsule of the invention, a gelling aid can also be used depending on the kind of gelling agent used. The following gelling aids can be used in combination with a carrageenan as the gelling agent. For κ-carrageenans, compounds that can yield one or more kinds of potassium ion, ammonium ion, and calcium ion in water, such as potassium chloride, ammonium chloride, ammonium acetate, and calcium chloride can be used. For ι-carrageenans, compounds that can yield a calcium ion in water, such as calcium chloride can be used. As gelling aids used in combination with a gellan gum as the gelling agent, compounds that can yield one or more kinds of sodium ion, potassium ion, calcium ionized, and magnesium ion in water, such as sodium chloride, potassium chloride, calcium chloride, and magnesium sulfate can be used. In addition, citric acid or sodium citrate can also be used as an organic acid or a water-soluble salt thereof.

A preferable combination is hydroxypropyl methylcellulose as a water-soluble cellulose compound, carrageenan as a gelling agent, and potassium chloride as a gelling aid.

In addition to the above-mentioned components, plasticizers; colorants such as dyes, pigments, and the like; opacifying agents; or flavoring agents can also be added to the hard capsule, as required.

Any plasticizers can be used without limitation insofar as they can be used for medical drugs or food products. For example, dioctyl adipate, polyester adipate, epoxidated soybean oil, epoxy hexahydro phthalic acid diester, kaolin, triethyl citrate, glycerol, glycerol fatty acid ester, sesame oil, a polydimethylsiloxane-silicon dioxide mixture, D-sorbitol, medium chain fatty acid triglyceride, sugar alcohol solution originated corn starch, triacetin, concentrated glycerin, castor oil, phytosterol, diethyl phthalate, dioctyl phthalate, dibutyl phthalate, butyl phthalyl butyl glycolate, propylene glycol, polyoxyethylene (105) polyoxy-propylene (5) glycol, polysorbate 80, macrogol 1500, macrogol 400, macrogol 4000, macrogol 600, macrogol 6000, isopropyl myristate, cotton seed oil-soybean oil mixture, glyceryl monostearate, isopropyl linolate, can be used as plasticizers.

Any colorants can be used without limitation insofar as they can be used for medical drugs or food products. For example, powdered catechutannic acid, turmeric extract, methylrosanilinium chloride, yellow iron oxide, yellow iron sesquioxide, OPASPRAY K-1-24904, orange essence, brown iron oxide, carbon black, caramel, carmine, carotene liquid, β-carotene, light sensitive element No. 201, licorice extract, gold leaf, sasa albomarginala extract, black iron oxide, light anhydrous silicic acid, kekketsu, zinc oxide, titanium oxide, iron sesquioxide, disazo yellow, food blue No. 1 and its aluminum lake, food blue No. 2 and its aluminum lake, food Yellow No. 4 and its aluminum lake, food Yellow No. 5 and its aluminum lake, food Green No. 3 and its aluminum lake, food red No. 2 and the aluminum lake, food red No. 3, food red No. 102 and its aluminum lake, food red No. 104 and its aluminum lake, food red No. 105 and its aluminum lake, food Red No106 and its aluminum lake, sodium hydroxide, talc, copper chlorofin sodium, copper chlorophyll, rye green leaf juice powder, rye green leaf extract, phenol red, sodium fluorescein, d-borneol, malachite green, octyl dodecyl myristate, methylene blue, medicinal carbon, riboflavin butyrate, riboflavin, powdered green tea, manganese ammonium phosphate, riboflavin sodium phosphate, rose oil, turmeric color, chlorophyll, carminic acid color, food red No. 40 and its aluminum lake, water-soluble annatto, sodium iron-chlorophyllin, dunaliella carotene, paprika colour, ginseng carotene, potassium norbixin, sodium norbixin, palm oil carotene, beat red, grape pericarp color, black currant color, monascus color, safflower red color, safflower yellow color, marigold color, sodium riboflavine phosphate, madder color, alkanet color, aluminum, potato carotene, shrimp color, krill color, orange color, cacao color, cacao carbon-dust color, oyster color, crab color, carob color, fish scale foil, silver, kusagi color, gardenia blue color, gardenia red color, gardenia yellow color and kooroo color, chlorophine, kaoliang color, bone char color, bamboo grass color, shea nut color, lithosperm root color, redsanders color, vegetable carbon black, sappan color, spirulina color, onion color, tamarind color, corn color, tomato color, peanut color, phaffia color, pecan nut color, monascus yellow, powdered annatto, hematococcus algae color, purple sweet potato color, purple corn color, purple yam color, vegetable oil soot color, lac color, rutin, enju extract, backwheat extract, logwood color, red cabegge color, red rice color, red color, azuki color, hydrangeae leaves extract, sepia color, uguisukagura color, elderberry color, olive tea, cowberry color, gooseberry color, cranberry color, salmon berry color, strawberry color, dark sweet cherry color, cherry color, thimbleberry color, deberry color, pineapple juice, huckleberry juice, grape juice color, black currant color, blackberry color, plum color, blueberry color, berry juice, boysenberry color, whortleberry color, mulberry color, morello cherry color, raspberry color, red currant color, lemon juice, loganberry color, powdered chlorella, cocoa, saffron color, beefsteak plant color, chicory color, layer color, hibiscus color, malt extract, powdered paprika, beet red juice, ginseng juice, can be used as colorants.

Any opacifying agents or flavoring agents can be used without limitation insofar as they can be used for medical drugs or food products. For example, as opacifying agents, titanium oxide, iron sesquioxide, yellow iron sesquioxide, black iron oxide, food blue No. 1 aluminum lake, food blue No. 2 aluminum lake, food yellow No. 4 aluminum lake, food yellow No. 5 aluminum lake, food green No. 3 aluminum lake, food red No. 2 aluminum lake, food red No. 3 aluminum Lake, food red No. 102 aluminum lake, food red No. 104 aluminum lake, food red No. 105 aluminum lake, food red No. 106 aluminum lake, and food red No. 40 aluminum lake can be used as opacifying agents.

The hard capsule of the invention is characterized, in that its capsule film has a low equilibrium moisture content. The equilibrium moisture of the capsule film can be evaluated from the moisture content of the film when a hard capsule is placed under a specific relative humidity condition. In particular, the hard capsule of the invention exhibits a loss on drying after 10 days of storage at 25° C. and at a relative humidity of 53% of less than 6% by weight. This is preferably 5.8% by weight or less, more preferably 5.5% by weight or less, and still more preferably 5% by weight or less.

The “loss on drying” of the invention means a decreased moisture content upon heating and drying a capsule film at 105° C. for 8 hours. The loss on drying after 10 days of storage at 25° C. and at a relative humidity of 53% can be measured by the method described below.

### Measurement Method for Loss on Drying

A sample (hard capsule) weighing 0.5 to 5.0 g is placed into a desiccator having an atmosphere in which the humidity is made constant by including a saturated aqueous solution of magnesium nitrate inside the desiccator, and then the desiccator is sealed and stored at 25° C. for 10 days. In the presence of a saturated aqueous solution of magnesium nitrate, the relative humidity can be adjusted to approximately 53%. The weight (wet weight) of the sample after storage is measured, and the sample is then heated at 105° C. for 8 hours. Then, the weight (dry weight) of the sample is measured again. From the difference in the weight of the sample between before drying (wet weight) and after drying (dry weight), the amount of moisture decrease (loss on drying) upon heating and drying at 105° C. for 8 hours is calculated according to the following equation.

Loss on drying(% by weight)=[(Wet weight of capsule−Dry weight of capsule)/Wet weight of capsule]×100 [Equation 1]

As for the hard capsule of the invention, the loss on drying after 10 days of storage at 25° C. and at a relative humidity of 12%, 22%, 33%, or 43% is 1.1% by weight or less, 2.1% by weight or less, 3.2% by weight or less, and 4.7% by weight or less, respectively. The hard capsule of the invention does not need to satisfy all of the loss-on-drying conditions at the above-mentioned relative humidity levels. Satisfying at least one of the conditions is sufficient. The hard capsule of the invention will preferably satisfy two or more conditions, more preferably three or more conditions, and still more preferably all four conditions.

The relative humidity conditions can each be attained using, in place of the saturated aqueous solution of magnesium nitrate, a saturated aqueous solution of lithium chloride, potassium acetate, magnesium chloride, or potassium carbonate in the above-mentioned method. More specifically, in the presence of the saturated aqueous solution of lithium chloride, potassium acetate, magnesium chloride, or potassium carbonate, the relative humidity can be set to approximately 12%, approximately 22%, approximately 33%, or approximately 43%, respectively.

The loss on drying after 10 days of storage at 25° C. and at a relative humidity of 12% is preferably 1% by weight or less, and more preferably 0.9% by weight or less; the loss on drying after 10 days of storage at 25° C. and at a relative humidity of 22% is preferably 1.9% by weight or less, and more preferably 1.6% by weight or less; the loss on drying after 10 days of storage at 25° C. and at a relative humidity of 33% is preferably 2.8% by weight or less, and more preferably 2.4% by weight or less; and the loss on drying after 10 days of storage at 25° C. and at a relative humidity of 43% is preferably 4.2% by weight or less, and more preferably 3.6% by weight or less.

Further, the hard capsule of the invention is characterized in that its capsule film has a low hygroscopicity.

The hygroscopicity of the capsule film can be evaluated from the relationship between relative humidity and the loss on drying (capsule moisture value (%)) of the capsule film at that relative humidity, as shown below.

### Method for Evaluating the Hygroscopicity of a Capsule Film

The moisture content of a sample (hard capsule) weighing 0.5 to 5.0 g is reduced with a silica gel, and the obtained sample is then placed into a desiccator, the desiccator having an atmosphere in which the humidity is made constant by including a saturated aqueous solution of lithium chloride, potassium acetate, magnesium chloride, potassium carbonate, magnesium nitrate, sodium chloride, or monobasic potassium phosphate inside the desiccator. Thereafter, the desiccator is sealed, and the sample is stored at 25° C. as is. In the presence of the saturated aqueous solution of lithium chloride, potassium acetate, magnesium chloride, potassium carbonate, magnesium nitrate, sodium chloride, or monobasic potassium phosphate, the relative humidity can be set to approximately 12%, approximately 22%, approximately 33%, approximately 43%, approximately 53%, approximately 75%, or approximately 96%, respectively.

The weight (wet weight) of the sample after storage is measured, and the sample is then heated at 105° C. for 8 hours. Subsequently, the weight (dry weight) of the sample is measured again. From the difference in the weight of the sample before drying (wet weight) and after drying (dry weight), the amount of moisture loss (loss on drying) upon heating and drying at 105° C. for 8 hours is calculated according to the following equation.

Capsule moisture value(%)=[(wet weight of capsule at a relative humidity−dry weight of capsule)/wet weight of capsule at a relative humidity]×100 [Equation 2]

Subsequently, the ratio (%) of the moisture value (%) of the capsule at a specific relative humidity (%) to the relative humidity (%) is calculated, and the hygroscopicity (%) of the capsule film is evaluated from this value.

Moisture absorption properties(%)=(capsule moisture value/the relative humidity)×100 [Equation 3]

Relative humidity: A %

Capsule moisture value: capsule moisture value (%) at A % relative humidity

More specifically, it is preferable that the hygroscopicity (%) of the hard capsule of the present invention at 25° C. satisfies at least one of the following conditions (1) to (5):

(1) 9.2% or less at a relative humidity of 12%;

(2) 9.5% or less at a relative humidity of 22%;

(3) 9.7% or less at a relative humidity of 33%;

(4) 10.9% or less at a relative humidity of 43%;

(5) 11.1% or less at a relative humidity of 53%.

The hygroscopicity (%) at 25° C. and at a relative humidity of 12% is preferably 8.3% or less and more preferably 7.5% or less. The hygroscopicity (%) at 25° C. and at a relative humidity of 22% is preferably 8.6% or less and more preferably 7.3% or less. The hygroscopicity (%) at 25° C. and at a relative humidity of 33% is preferably 8.5% or less and more preferably 7.3% or less. The hygroscopicity (%) at 25° C. and at a relative humidity of 43% is preferably 9.8% or less and more preferably 8.4% or less. The hygroscopicity (%) at 25° C. and at a relative humidity of 53% is preferably 10.4% or less and more preferably 9.4% or less.

The hard capsule of the invention having a low moisture content and a low hygroscopicity can be produced by a dip coating method. Specifically, such a hard capsule can be obtained by dipping a capsule-mold pin into an aqueous solution comprising the above-described components as a dipping solution (hereinafter referred to as “capsule-preparing solution”);

drawing the capsule-mold pin out of the solution; and gelling the solution that is adhering to the outside surface of the capsule-mold pin at a temperature of 35° C. or less.

The concentration of each of the above-mentioned components contained in the capsule-preparing solution is not limited, and the following proportions can be mentioned.

The water-soluble cellulose compound is 5 to 30% by weight, preferably 10 to 28% by weight, and more preferably 16 to 24% by weight. The gelling agent is 0.01 to 0.5% by weight, preferably 0.02 to 0.45% by weight, and more preferably 0.03 to 0.4% by weight. The gelling aid, if added, is 0.01 to 0.5% by weight, preferably 0.02 to 0.45% by weight, and more preferably 0.03 to 0.4% by weight.

The amount of water contained in the capsule-preparing solution is not limited, and is adjusted in such a manner that the viscosity of the capsule-preparing solution is 100 to 20000 mPa·s and preferably 300 to 10000 mPa·s at a temperature (30 to 80° C. and preferably 40 to 60° C.) for dipping the capsule-mold pin (dipping solution temperature). In usual, the water content is 70 to 95% by weight and preferably 72 to 90% by weight.

The method for producing the capsule-preparing solution (dipping solution) is not limited, and various methods can be employed. For example, the following methods can be mentioned: a method comprising dissolving a gelling agent and, if needed, a gelling aid in purified water heated at approximately 70° C., dispersing a water-soluble cellulose compound into the purified water, and cooling the result to the desired temperature (usually 30 to 80° C., preferably 40 to 60° C., and more preferably 50 to 60° C.) for the dipping solution; and a method comprising dispersing a water-soluble cellulose compound into hot water of approximately 70° C. or above, cooling the hot water to approximately 35° C. or less to dissolve the water-soluble cellulose compound to yield a solution, warming the solution to approximately 35 to 50° C., adding and dissolving a gelling agent to the warmed solution, and adjusting the temperature of the result to a desired temperature for a dipping solution.

The hard capsule of the invention is produced by dipping a capsule-mold pin into the capsule-preparing solution (dipping solution), drawing the capsule-mold pin out of the solution, gelling, by cooling, the capsule-preparing solution that is adhering to the outside surface of the capsule-mold pin at a temperature of 35° C. or less, and heating the gelled capsule film to 50 to 150° C.

Specifically, the hard capsule of the invention can be produced by the following process:

(1) a step of dipping a capsule-mold pin into a capsule-preparing solution comprising a water-soluble cellulose compound, a gelling agent, and, if required, a gelling aid (dipping step);

(2) a step of drawing the capsule-mold pin out of the capsule-preparing solution, and gelling, by cooling, the solution that is adhering to the outside surface of the capsule-mold pin to 35° C. or less (gelling step);

(3) a step of drying the gelled capsule film formed in covering the outside surface of the capsule-mold pin (drying step);

(4) a step of removing the dried capsule film from the capsule-mold pin (removing step), and

(5) a step of heating the gelled and/dried capsule film to 50 to 150° C.; after step (2); before, after, or simultaneously with step (3); or after step (4) (heating step).

In the dipping step (1), the temperature of the capsule-preparing solution (dipping solution) is ordinarily 30 to 80° C., preferably 40 to 60° C., and more preferably 50 to 60° C. The temperature of the capsule-mold pin to be dipped in the capsule-preparing solution (dipping solution) is determined according to the temperature of the capsule-preparing solution, and is ordinarily 10 to 30° C., preferably 13 to 28° C., and more preferably 15 to 25° C.

The capsule-preparing solution used in the invention usually has a property of turning into a gel 35° C. or less (cold gelling). Therefore, the gelling step (2) can be performed by allowing the capsule production room containing the capsule-preparing solution to cool to 35° C. or less, preferably 30° C. or less, and more preferably room temperature or less (gel cooling process).

The drying step (3) can be performed at room temperature. Ordinarily, the drying step is carried out by blowing room temperature air. The removing step (4) is performed by removing the dried capsule film formed on the surface of the capsule-mold pin from the pin.

The heating step (5) is performed after the gelling step (2) (i.e., after gelling the capsule-preparing solution). The heating step may be performed at any stage after the gelling step (2), and may be performed before or after the drying step (3). Alternatively, the drying step and the heating step may be performed simultaneously. The heating step may be performed after the removing step (4). It is preferable that a gelled capsule film be dried at room temperature after the gelling step (2), and be heated after being dried or half-dried.

The heating temperature is not limited insofar as it ranges from 50 to 150° C., and the heating temperature is preferably 60 to 100° C., and more preferably 60 to 80° C. Ordinarily, the heating step is carried out by blowing air of 50 to 150° C.

The capsule film thus obtained is cut to a predetermined length to produce a hard capsule of the invention having low moisture content and low hygroscopicity wherein a body part and a cap part are coupled or not coupled.

The capsule of the invention prepared by the above-described method contains a water-soluble cellulose compound in a proportion of 70 to 99.9% by weight, preferably 75 to 99.7% by weight, more preferably 80 to 99.4% by weight, and still more preferably 85 to 99% by weight. The capsule of the invention prepared by the above-described method contains a gelling agent in a proportion of 0.05 to 10% by weight, preferably 0.1 to 9.5% by weight, more preferably 0.2 to 9% by weight, and more preferably 0.3 to 8% by weight. When a gelling aid, such as potassium chloride, is used, the content of the gelling aid, for example, is in the range of 2.2% by weight or less, preferably 0.1 to 2.1% by weight, more preferably 0.2 to 1.9% by weight, and still more preferably 0.3 to 1.6% by weight. When the capsule of the invention contains a plasticizer, the content thereof is ordinarily, for example, in the range of 15% by weight or less, preferably 13% by weight or less, more preferably 11% by weight or less, and still more preferably 8% by weight or less. When a coloring agent is used, the content thereof can be suitably determined according to the desired coloring grade in the range of 15% by weight or less, preferably 13% by weight or less, more preferably 11% by weight or less, and still more preferably 8% by weight or less.

### II. Capsule Product

The above-described hard capsule of the invention can be prepared as a capsule product by inserting drugs, food products, or cosmetic materials into the capsule.

Any components can be inserted into the capsule without limitation insofar as they do not dissolve the film of the hard capsule of the invention, and do not react with the film. For example, liquefied materials or gelatinous materials in addition to solid materials in the form of powders, granules, etc., can be inserted therein. Examples of such liquefied materials include alcohols such as stearyl alcohol, cetanol, polyethylene glycol 600, polyethylene glycol 800, polyethylene glycol 1000, polyethylene glycol 1500, polyethylene glycol 2000, polyethylene glycol 3000, polyethylene glycol 4000, polyethylene glycol 6000, polyethylene glycol 8000, polyethylene glycol 20000, etc.; fats and oils, such as sesame oil, soybean oil, arachis oil, corn oil, hardened oil, paraffin oil, white beeswax, etc.; fatty acids, such as stearic acid, palmitic acid, myristic acid, triethyl citrate, triacetone, medium chain fatty acid triglyceride, etc.; and derivatives thereof. Ordinarily, these liquefied materials are inserted into the above-described hard capsule of the invention in the form of a mixture with active ingredients or principal components such as drugs, food products, or cosmetic materials.

Drugs inserted into the hard capsule of the invention are not limited, and those that can be administered orally can be mentioned as a main example. For example, such orally administered drugs include vitamins, antipyretics, analgesics, antiphlogistics, antitumor agents, cardiotonics, anticoagulants, hemostats, bone-resorption inhibitors, vascularization inhibitors, antidepressants, antitumor agents such as proton pump inhibitors (e.g., benzimidazole derivatives), antiussive/expectorant agents, antiepileptic agents, antiallergic agents, antiarrhythmics, vasodilators, hypotensive diuretics, diabetic medicines, antituberculous agens, hormones, antinarcotic agents, etc.

The above-mentioned drugs are inserted into the hard capsule of the invention by, for example, known encapsulation machines, such as fully automatic encapsulation machines and encapsulationsealing•machines. A fully automatic encapsulation machine manufactured by Qualicaps (model name: LIQFIL super 80/150) can be mentioned as an example of the former, and an encapsulationsealing machine manufactured by Qualicaps (model name: LIQFIL super FS) can be mentioned as an example of the latter.

Since the film of the hard capsule of the invention is low in both moisture content and hygroscopicity as described above, the hard capsule of the invention can be filled with components having relatively high reactivity to moisture (e.g., components that easily deteriorate on contact with moisture), and therefore can be used in capsule products. Ester compounds and enzymes, for example, can be mentioned as such components. The hard capsule of the invention exhibits excellent strength (impact resistance) under low moisture conditions. Therefore, in the case where a component with high reactivity to moisture or high hygroscopicity is inserted into the capsule of the invention, deterioration of the inserted component can be prevented by storing the hard capsule under dry conditions with capsule resistibility remaining. Since the film of the hard capsule of the invention has low hygroscopicity, a component with a relatively high moisture content can be inserted therein. Components exhibiting considerable hydration, such as enzymes and morphines, can be mentioned as such components.

### Ill. Method for Reducing Moisture Content and Hygroscopicity for the Film of a Hard Capsule

The invention provides a method for reducing the moisture content and hygroscopicity of the film of a hard capsule comprising as principal components a water-soluble cellulose compound, a gelling agent, and, if required, a gelling aid. The target hard capsule of the invention is prepared by a gel cooling process using the above-mentioned components, and the following steps:

(a) a step of dipping a capsule-mold pin into a capsule-preparing solution comprising a water-soluble cellulose compound, a gelling agent, and, if required, a gelling aid (dipping step);

(b) a step of drawing the capsule-mold pin out of the capsule-preparing solution, and gelling the solution that is adhering to the outside surface of the capsule-mold pin at 35° C. or less (gelling step);

(c) a step of drying the gelled capsule film formed in covering the outside surface of the capsule-mold pin (drying step); and

(d) a step of removing the dried capsule film from the capsule-mold pin (removing step).

The kinds and content of water-soluble cellulose compounds, gelling agents, and gelling aids and the preparation method of the capsule-preparing solution containing these are the same as mentioned in paragraph I. The steps (a), (b), (c), and (d) correspond to the steps (1), (2), (3), and (4) of the above-described method for producing a hard capsule of the invention, respectively.

The production method comprising steps (a) to (d) is equivalent to hitherto-known methods for producing a cold-gel hard capsule that comprises as principal components a water-soluble cellulose compound, a gelling agent, and, if required, a gelling aid, and that is produced by the gel cooling process. According to hitherto-known gel cooling processes, the capsule-preparing solution is gelled at 35° C. or less, and subsequently the result is dried at room temperature or less, thereby yielding a hard capsule (cold gel hard capsule).

The production method of the invention can be effected by heating a gelled capsule film to 50 to 150° C.; after the above-described gelling step (b); or before, after or simultaneously with the drying step (c); or after the removing step (d).

The temperature for the heating step is not limited, insofar as the temperature is in the range of 50 to 150° C. The heating temperature is preferably in the range of 60 to 100° C., and more preferably 60 to 80° C. Ordinarily, the heating step is effected by blowing air in the above-mentioned temperature ranges, but is not limited thereto.

The drying step (c) may be ordinary carried out by blowing air in room temperature.

As described above, the invention makes it possible to reduce the moisture content and hygroscopicity of the film of a hard capsule (cold-gel hard capsule) that comprises as principal components a water-soluble cellulose compound, a gelling agent, and, if required, a gelling aid, and that is produced by a gel cooling process. More specifically, the invention can provide, according to the above-described method, a cold-gel hard capsule whose film has a lower moisture content and lower hygroscopicity than those of hitherto-known hard capsules (hereinafter sometimes referred to as a hitherto-known cold-gel hard capsule) produced by a gel cooling process comprising the steps (a) to (d).

The loss on drying of the hitherto-known cold gel hard capsules after 10 days of storage at 25° C. and at a relative humidity of 53% is in the range of 6% by weight or more, particularly in the range of 6 to 7% by weight (6.7% by weight in one example). In contrast, according to the method of the invention, the loss on drying can be reduced to 6% by weight or less, preferably 5.8% by weight or less, more preferably 5.5% by weight or less, and still more preferably 5% by weight or less, and thus a hard capsule with a low moisture content can be obtained (See Experimental Examples). The loss on drying of the hitherto-known cold gel hard capsules is as follows:

the loss on drying after 10 days of storage at a relative humidity of 12% is approximately 1.3% by weight;

the loss on drying after 10 days of storage at a relative humidity of 22% is approximately 2.4% by weight;

the loss on drying after 10 days of storage at a relative humidity of 33% is approximately 3.6% by weight; and

the loss on drying after 10 days of storage at a relative humidity of 43% is approximately 5.3% by weight.

In contrast, the method of the invention can provide a cold-gel hard capsule with a low moisture content satisfying at least one of the following conditions:

the loss on drying after 10 days of storage at a relative humidity of 12% is 1.1% by weight or less, preferably 1% by weight or less, and more preferably 0.9% by weight or less;

the loss on drying after 10 days of storage at a relative humidity of 22% is 2.1% by weight or less, preferably 1.9% by weight or less, and more preferably 1.6% by weight or less;

the loss on drying after 10 days of storage at a relative humidity of 33% is 3.2% by weight or less, preferably 2.8% by weight or less, and more preferably 2.4% by weight or less; and

the loss on drying after 10 days of storage at a relative humidity of 43% is 4.7% by weight or less, preferably 4.2% by weight or less, and more preferably 3.6% by weight or less.

The method of the invention also can make it possible to reduce the hygroscopicity of the film of a hard capsule to produce a low hygroscopic hard capsule as described below (see Experimental Examples):

(1) hygroscopicity (%) at 25° C. and at a relative humidity of 12% is 9.2% or less, preferably 8.3% or less, and more preferably 7.5% or less;

(2) hygroscopicity (%) at 25° C. and at a relative humidity of 22% is 9.5% or less, preferably 8.6% or less, and more preferably 7.3% or less;

(3) hygroscopicity (%) at 25° C. and at a relative humidity of 33% is 9.7% or less, preferably 8.5% or less, and more preferably 7.3% or less;

(4) hygroscopicity (%) at 25° C. and at a relative humidity of 43% is 10.9% or less, preferably 9.8% or less, and more preferably 8.4% or less; or

(5) hygroscopicity (%) at 25° C. and at a relative humidity of 53% is 11.1% or less, preferably 10.4% or less, and more preferably 9.4% or less.

### EXAMPLES

The present invention is further described below by means of Examples and Experimental Examples, which are not intended to limit the scope of the disclosure.

### Example 1

To 19.55 L of purified water at about 70° C. was added 18.4 g of potassium chloride (gelling aid) and dissolved. To the mixture was further added 39.1 g of Kappa carrageenan (gelling agent) and dissolved with stirring. Then, 3.45 kg of hydroxypropyl methylcellulose (cellulose derivative) was poured into the solution with stirring, and dispersed in the hot water. The solution temperature was then lowered to 50° C., and the hydroxypropyl methylcellulose was dissolved with stirring. The resulting solution was allowed to stand for 7 hours for degassing.

The capsule-preparing solution thus obtained was used as a dipping solution for making hard capsules by means of a gel cooling process. Specifically, capsule-mold pins at about 20° C. were dipped into the capsule-preparing solution (dipping solution) at 45 to 55° C. The pins were subsequently withdrawn from the dipping solution, and cooled at room temperature for 20 to 90 seconds. The capsule-preparing solution (dipping solution) that adhered to the outer surface of each pin was allowed to gel to form a film. The film was further left standing at room temperature for 5 to 20 minutes and dried, followed by heat drying. After the heat drying, the capsule film thus formed was stripped from the pin, and cut to a predetermined length. A body portion and a cap portion were then coupled to obtain a hard capsule. The conditions for the above-described dry heating were varied as follows: 30 minutes at 50° C. in Formulation 1, 30 minutes at 60° C. in Formulation 2, 30 minutes at 70° C. in Formulation 3, 30 minutes at 80° C. in Formulation 4, 30 minutes at 90° C. in Formulation 5, 30 minutes at 100° C. in Formulation 6, 30 minutes at 110° C. in Formulation 7, 30 minutes at 120° C. in Formulation 8, and 30 minutes at 150° C. in Formulation 9.

### Example 2

To 19.55 L of purified water at about 70° C. was added 18.4 g of potassium chloride (gelling aid) and dissolved. To the mixture was further added 39.1 g of Kappa carrageenan (gelling agent) and dissolved with stirring. Then, 3.45 kg of hydroxypropyl methylcellulose (cellulose derivative) was poured into the solution with stirring, and dispersed in the hot water. The solution temperature was then lowered to 50° C., and the hydroxypropyl methylcellulose was dissolved with stirring. The resulting solution was allowed to stand for 7 hours for degassing.

The capsule-preparing solution thus obtained was used as a dipping solution for making hard capsules by means of a gel cooling process. Specifically, capsule-mold pins at about 20° C. were dipped into the capsule-preparing solution (dipping solution) at 45 to 55° C. The pins were subsequently withdrawn from the dipping solution, and cooled at room temperature for 20 to 90 seconds. The capsule-preparing solution (dipping solution) that adhered to the outer surface of each pin was allowed to gel to form a film. The film was further left standing at room temperature for 40 to 90 minutes and dried. The capsule film thus formed was then withdrawn from the pins, and cut to a predetermined length. A body portion and a cap portion were coupled, and subsequently heated to obtain a hard capsule. The conditions for the above-described heat treatment were varied as follows: 1 hour at 50° C. (Formulation 10), and 1 hour at 70° C. (Formulation 11).

### Experimental Example 1

### Evaluation of the Moisture Content of Capsule Films

The hard capsules according to Formulations 1 to 9 prepared in Example 1 above, along with hard capsules prepared by drying at room temperature without heating (control sample), were left standing at 25° C. and at a relative humidity of 53% for 10 days, i.e., until the moisture of the capsule films reached equilibrium. The wet weight of each of the resulting hard capsules of Formulations 1 to 9 and the control sample was measured, and then the capsules were heat-treated at 105° C. for 8 or 24 hours to measure the dry weight. The loss on drying (%) of each capsule after drying was determined in accordance with Equation 4 by subtracting the dry weight from the wet weight. The equilibrium moisture content of each capsule film was thus evaluated based on its loss on drying (%).

Loss on drying(% by weight)=[(wet weight−dry weight)/wet weight]×100 [Equation 4]

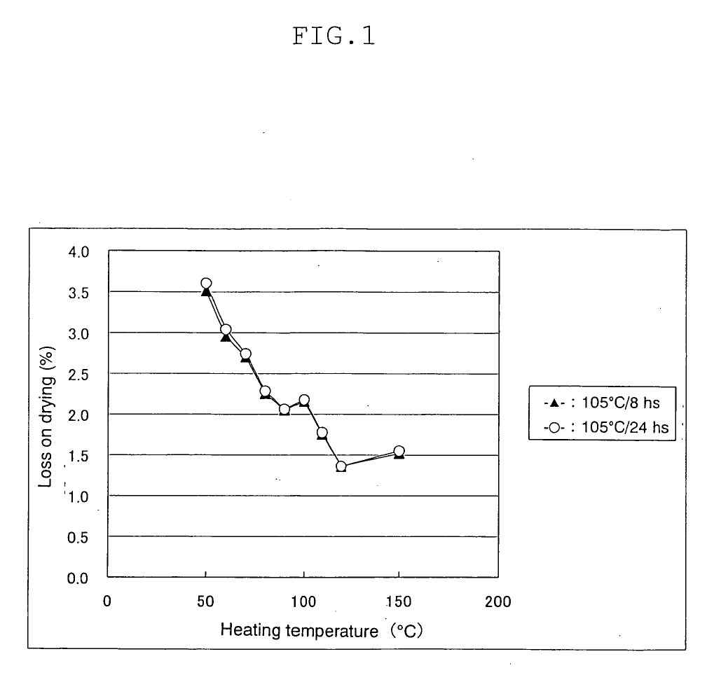

Table 1 and FIG. 1 show the loss on drying (%) of the hard capsules of Formulations 1 to 9 and the control sample.

TABLE 1

| | Heating | loss on drying (% by wt) | |
|---|---|---|---|
| | temperature (° C.) | 105° C./8 hrs | 105° C./24 hrs |
| Formulation 1 | 50 | 3.51 | 3.61 |
| Formulation 2 | 60 | 2.96 | 3.04 |
| Formulation 3 | 70 | 2.70 | 2.74 |
| Formulation 4 | 80 | 2.26 | 2.29 |
| Formulation 5 | 90 | 2.05 | 2.06 |
| Formulation 6 | 100 | 2.16 | 2.18 |
| Formulation 7 | 110 | 1.76 | 1.78 |
| Formulation 8 | 120 | 1.36 | 1.36 |
| Formulation 9 | 150 | 1.52 | 1.55 |
| Control sample | Not heated | 6.48 | 6.52 |

The results establish that the hard capsules prepared according to the method of the invention have lower moisture content (equilibrium moisture content) than that of the hitherto-known cold-gel hard capsules prepared by the conventional gel cooling process.

### Experimental Example 2

### Evaluation of Moisture Absorption/Release Properties of Capsule Films Using a Microbalance

The hard capsules prepared according to Formulation 11 (heated at 70° C. for 1 hour) in Example 2 above, along with the hard capsules prepared as a control sample by drying at room temperature without heating, were evaluated for their moisture absorption/release properties.

### (1) Moisture Adsorption

To the cell of a microbalance (MB 300G, manufactured by VTI Corporation) was placed each sample (hard capsules: 5-10 mg). The cell was then evacuated at 25° C. using a vacuum pump to make the moisture content of the hard capsules 0%. The weight of the sample with no moisture content was measured to determine the dry weight. The relative humidity of the cell was subsequently increased from a relative humidity of 0 to about 95% in increments of about 5%, and the weight of the sample was measured at each relative humidity (wet weight). The mass change (wet weight−dry weight) was determined as the moisture content, and the moisture value (%) of the capsule, that is, the moisture adsorption value (%), was determined in accordance with the following Equation 5:

Capsule moisture value(%)=[(wet weight at each relative humidity−dry weight)/wet weight at each relative humidity]×100 [Equation 5]

### (2) Moisture Desorption

When the relative humidity had reached about 95%, the relative humidity was then decreased from about 95% to about 5% in increments of about 5%, and the weight of each sample was measured at each relative humidity (wet weight). The mass change (wet weight−dry weight) was determined, and then the capsule moisture value (%), that is, the moisture desorption value (%), was determined in the same manner as described above.

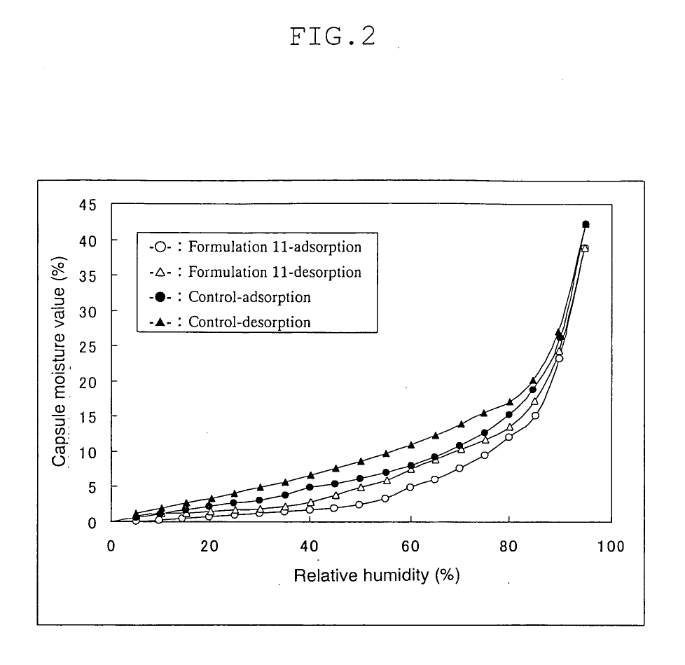

The results are shown in Table 2. FIG. 2 also shows the moisture adsorption/desorption isotherms of moisture adsorption/desorption to/from the capsule films, wherein the horizontal axis represents relative humidity (%), and the vertical axis represents capsule moisture value (%).

TABLE 2

| Formulation 11 | | | | | | | |
|---|---|---|---|---|---|---|---|
| (heart-treated at 70° C. for 1 hour) | | | | Control sample | | | |
| Adsorption | | Desorption | | Adsorption | | Desorption | |
| | Moisture | | Moisture | | Moisture | | Moisture |
| RH (%) | value (%) | RH (%) | value (%) | RH (%) | value (%) | RH (%) | value (%) |
| −0.11 | 0 | 94.904 | 38.904 | −0.093 | 0 | 94.965 | 42.289 |
| 5.166 | 0.105 | 89.942 | 24.452 | 5.146 | 0.645 | 89.777 | 27.092 |
| 9.838 | 0.303 | 85.089 | 17.263 | 10.018 | 1.119 | 84.799 | 20.22 |
| 14.349 | 0.522 | 80.025 | 13.59 | 15.023 | 1.731 | 80.18 | 17.158 |
| 19.809 | 0.733 | 75.185 | 11.738 | 19.814 | 2.158 | 74.838 | 15.562 |
| 24.958 | 0.993 | 70.23 | 10.309 | 24.937 | 2.66 | 70.083 | 13.89 |
| 30.052 | 1.18 | 65.021 | 8.936 | 30.009 | 3.117 | 65.09 | 12.375 |
| 34.938 | 1.421 | 60.087 | 7.509 | 35.113 | 3.769 | 60.101 | 11.002 |
| 39.901 | 1.721 | 55.2 | 5.882 | 39.983 | 4.886 | 54.996 | 9.749 |
| 44.803 | 2.003 | 50.106 | 4.962 | 45.002 | 5.455 | 50.177 | 8.624 |
| 49.781 | 2.463 | 45.172 | 3.775 | 49.908 | 6.164 | 45.119 | 7.586 |
| 54.881 | 3.278 | 40.201 | 2.849 | 55.061 | 7.017 | 40.182 | 6.614 |
| 59.816 | 4.881 | 35.231 | 2.275 | 60.003 | 8.068 | 35.254 | 5.706 |
| 64.808 | 5.982 | 30.18 | 1.864 | 64.817 | 9.281 | 30.184 | 4.882 |
| 69.811 | 7.681 | 25.175 | 1.692 | 69.797 | 10.803 | 25.131 | 4.123 |
| 74.891 | 9.5045 | 20.242 | 1.525 | 74.908 | 12.723 | 20.339 | 3.381 |
| 80.004 | 12.074 | 15.227 | 1.252 | 79.882 | 15.307 | 15.202 | 2.669 |
| 85.178 | 15.202 | 10.235 | 1.175 | 84.692 | 18.854 | 10.251 | 1.972 |
| 89.83 | 23.245 | 5.251 | 0.882 | 90.016 | 26.292 | 5.228 | 1.256 |
| 94.904 | 38.904 | | | 94.965 | 42.289 | | |

The results establish that the hard capsules prepared according to the method of the invention (Formulation 11) have lower moisture absorption/release properties than those of the hitherto-known cold-gel hard capsules prepared by the conventional gel cooling process, and are thereby capable of maintaining lower moisture content even at the same relative humidity.

### Experimental Example 3

### Evaluation of Moisture Absorption/Release Properties of Capsule Films Using Saturated Aqueous Salt Solutions

The hard capsules prepared according to Formulations 10 and 11 in Example 2 above, along with the hard capsules prepared as a control sample by drying at room temperature without heating, were evaluated for their moisture absorption/release properties.

### (1) Moisture Adsorption Experiment

After the moisture content of each of the hard capsules (samples) was reduced with silica gel, the sample was sealed in a container including a saturated aqueous solution of lithium chloride, potassium acetate, magnesium chloride, potassium carbonate, magnesium nitrate, sodium chloride or potassium dihydrogen phosphate and having a constant humidity. The sample was then kept in the container for 10 days. Lithium chloride, potassium acetate, magnesium chloride, potassium carbonate, magnesium nitrate, sodium chloride and potassium dihydrogen phosphate create atmospheres having relative humidities of about 12, 22, 33, 43, 53, 75 and 96%, respectively. After storage, the wet weight of each sample was measured, and then the sample was dry-heated at 105° C. for 8 hours to measure the dry weight. The reduction in the percentage of moisture content for each sample after heat-drying at 105° C. for 8 hours (i.e., loss on drying %) was determined from the difference between the wet weight before drying and the dry weight after drying, in accordance with the following Equation 6. The value obtained is denoted herein as the “capsule moisture value (%)”, that is, the moisture adsorption value (%).

Capsule moisture value(%)=[(wet weight at each relative humidity−dry weight)/wet weight at each relative humidity]×100 [Equation 6]

### (2) Moisture Desorption Experiment

About 10 g each of the hard capsules prepared according to Formulations 10 and 11, along with about 10 g of the hard capsules as a control sample, were individually sealed in a container including a saturated aqueous solution of potassium dihydrogen phosphate, and then the moisture content of each sample was increased. The sample was then placed into a different container including a saturated aqueous solution of lithium chloride, potassium acetate, magnesium chloride, potassium carbonate, magnesium nitrate or sodium chloride and having a constant humidity, and kept for 10 days. After storage, the wet weight of each sample was measured, and then the sample was dry-heated at 105° C. for 8 hours to measure the dry weight. The capsule moisture value (%), i.e., moisture desorption value (%), was determined from the difference between the wet weight before drying and dry weight after drying, in accordance with the above-described Equation 6.

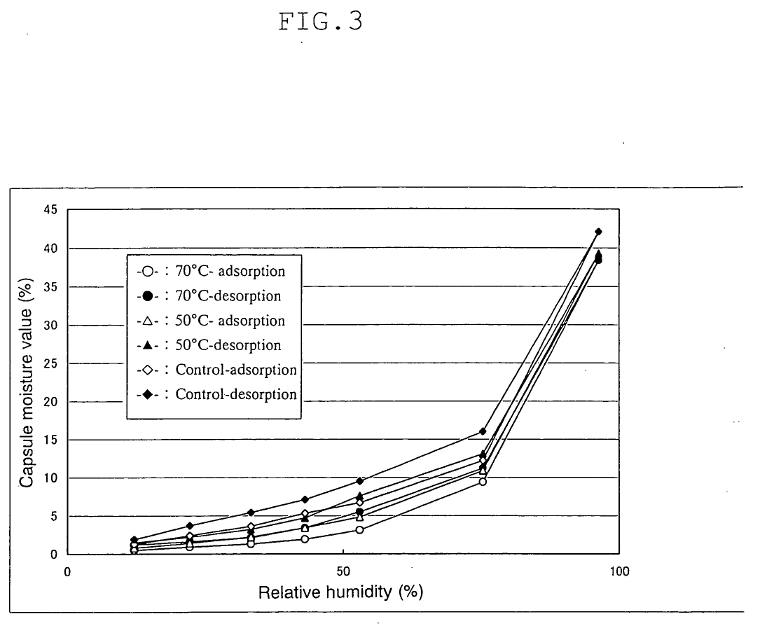

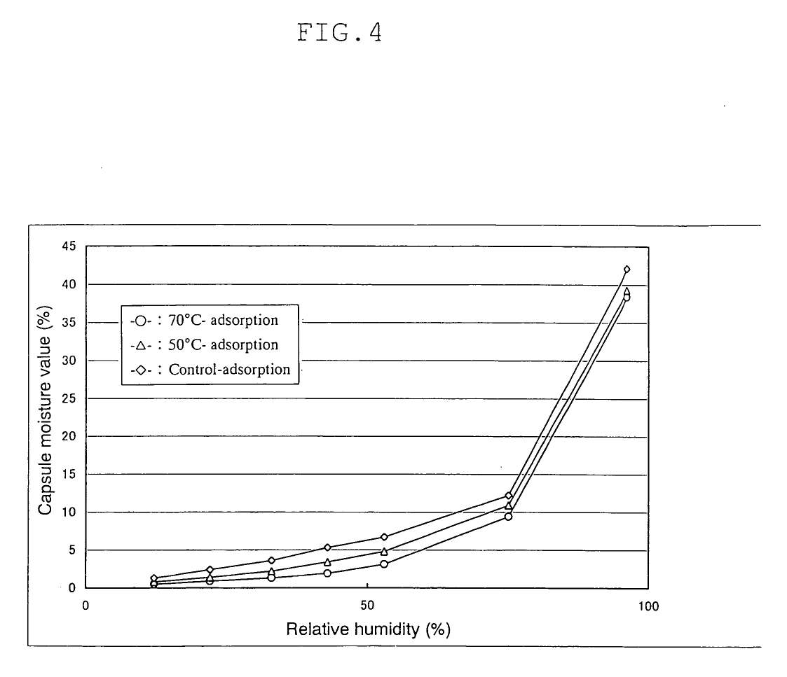

The results are shown in Table 3, in which “RH” denotes relative humidity, and “moisture value” denotes the capsule moisture value. FIG. 3 and FIG. 4 each show the moisture adsorption/desorption isotherms of moisture adsorption/desorption to/from the capsule films, wherein the horizontal axis represents relative humidity (%), and the vertical axis represents the capsule moisture value (%). FIG. 4 shows only the moisture adsorption isotherms taken from FIG. 3.

TABLE 3

| Formulation 11 | | | | Formulation 10 | | | | | | | |
|---|---|---|---|---|---|---|---|---|---|---|---|
| (heat-treated at 70° C. for 1 hr) | | | | (heat-treated at 50° C. for 1 hr) | | | | Control sample (not heated) | | | |
| Adsorption | | Desorption | | Adsorption | | Desorption | | Adsorption | | Desorption | |
| RH | Moisture | RH | Moisture | RH | Moisture | RH | Moisture | RH | Moisture | RH | Moisture |
| (%) | value (%) | (%) | value (%) | (%) | value (%) | (%) | value (%) | (%) | value (%) | (%) | value (%) |
| 12 | 0.5 | 96 | 38.4 | 12 | 0.8 | 96 | 39.3 | 12 | 1.3 | 96 | 42.1 |
| 22 | 0.9 | 75 | 11.2 | 22 | 1.4 | 75 | 13.1 | 22 | 2.4 | 75 | 16.0 |
| 33 | 1.3 | 53 | 5.5 | 33 | 2.2 | 53 | 7.6 | 33 | 3.6 | 53 | 9.5 |
| 43 | 1.9 | 43 | 3.4 | 43 | 3.4 | 43 | 4.7 | 43 | 5.3 | 43 | 7.1 |
| 53 | 3.1 | 33 | 2.1 | 53 | 4.8 | 33 | 3.2 | 53 | 6.7 | 33 | 5.4 |
| 75 | 9.4 | 22 | 1.6 | 75 | 10.9 | 22 | 2.2 | 75 | 12.2 | 22 | 3.7 |
| 96 | 38.4 | 12 | 1.2 | 96 | 39.3 | 12 | 1.5 | 96 | 42.1 | 12 | 1.9 |

The moisture absorption properties (%) of each capsule film were then evaluated by determining the percentage of the capsule moisture value at each specific relative humidity (%) obtained from the moisture adsorption experiment to the relative humidity (%), in accordance with the following Equation 7.

Moisture absorption properties(%)=(capsule moisture value/relative humidity)×100 [Equation 7]

Relative humidity: A %
Capsule moisture value: capsule moisture value (%) at A % relative humidity

The results are shown in Table 4.

TABLE 4

| Formulation 11 | | | Formulation 10 (heat-treated | | | Control sample | | |
|---|---|---|---|---|---|---|---|---|
| (heat-treated at 70° C. for 1 hr) | | | at 50° C. for 1 hr) | | | (not heated) | | |
| Adsorption | | | Adsorption | | | Adsorption | | |
| | Moisture | Moisture | | Moisture | Moisture | | Moisture | Moisture |
| RH | value | adsorption | RH | value | adsorption | RH | value | adsorption |
| (%) | (%) | properties | (%) | (%) | properties | (%) | (%) | properties |
| 12 | 0.5 | 4.166 | 12 | 0.8 | 6.666 | 12 | 1.3 | 10.833 |
| 22 | 0.9 | 4.090 | 22 | 1.4 | 6.363 | 22 | 2.4 | 10.909 |
| 33 | 1.3 | 3.939 | 33 | 2.2 | 6.666 | 33 | 3.6 | 10.909 |
| 43 | 1.9 | 4.418 | 43 | 3.4 | 7.906 | 43 | 5.3 | 12.325 |
| 53 | 3.1 | 5.849 | 53 | 4.8 | 9.056 | 53 | 6.7 | 12.641 |
| 75 | 9.4 | 12.533 | 75 | 10.9 | 14.533 | 75 | 12.2 | 16.266 |
| 96 | 38.4 | 40.000 | 96 | 39.3 | 40.937 | 96 | 42.1 | 43.854 |

The results establish that the hard capsules obtained according to the method of the invention (Formulations 10 and 11) have lower moisture absorption properties than those of the control hard capsules prepared by the conventional gel cooling process, and are thereby capable of maintaining lower moisture content even at the same relative humidity.

Accordingly, the results obtained from Experimental Examples 1, 2 and 3 reveal that the hard capsules of the invention are low in moisture content and moisture absorption/release properties, and are therefore suitable for use as a capsule filled with a drug, food ingredient or other material that is highly reactive with moisture and easily deteriorates, or with a drug, food ingredient or other material having high moisture absorption properties.

### Experimental Example 4

### Evaluation of Capsule Film Strength

The hard capsules prepared according to Formulation 10 (heat-treated at 50 (C for 1 hour) and Formulation 11 (heat-treated at 70 (C for 1 hour) in Example 2 above, along with gelatin hard capsules as a comparative example and hitherto-known cold-gel hard capsules prepared as a control sample by drying at room temperature without heating, were measured for their shock resistance in the manner described below.

### (1) Shock Resistance

Empty hard capsules of each of the aforementioned types having a different moisture content were placed horizontally, and a 50 g weight was dropped from a height of 10 cm. The failure rate was determined from the number of broken capsules. Testing was done 10 times.

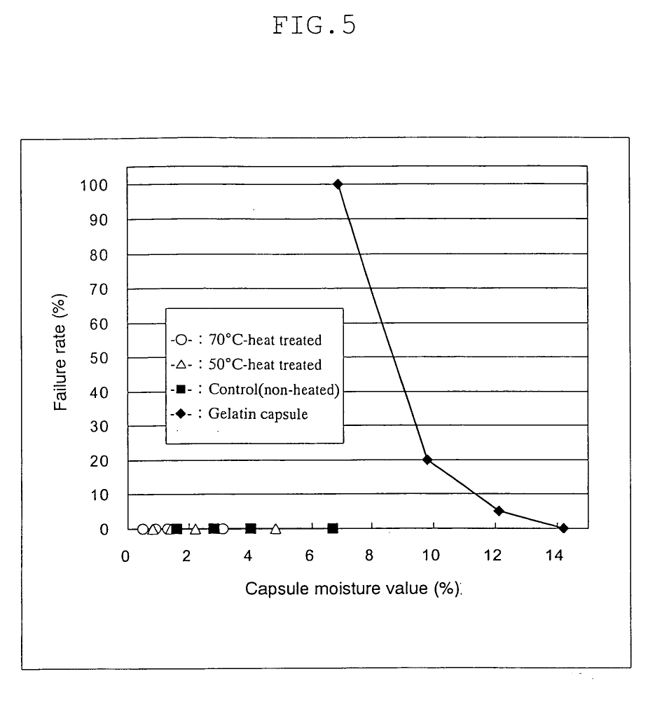

Table 5 and FIG. 5 show the results of the shock resistance tests.

TABLE 5

| | | | | | |
|---|---|---|---|---|---|
| Formulation 11 | Moisture value (%) | 0.5 | 0.9 | 1.3 | 3.1 |
| (heat-treated | Failure rate (%) | 0 | 0 | 0 | 0 |
| at 70° C. for 1 hr) | | | | | |
| Formulation 10 | Moisture value (%) | 0.8 | 1.4 | 2.2 | 4.8 |
| (heat-treated | Failure rate (%) | 0 | 0 | 0 | 0 |
| at 50° C. for 1 hr) | | | | | |
| Control sample | Moisture value (%) | 1.6 | 2.8 | 4.0 | 6.7 |
| (not heat-treated) | Failure rate (%) | 0 | 0 | 0 | 0 |
| Comparative example | Moisture value (%) | 6.9 | 9.8 | 12.1 | 14.2 |
| (Gelatin capsule) | Failure rate (%) | 100 | 20 | 5 | 0 |

The results establish that the hard capsules of the invention exhibit high strength even with a low moisture content, whereas the gelatin capsules are more likely to break with a lower moisture content.

### Examples 3 to 13

Low-moisture-content hard capsules in accordance with the invention having the compositions listed in Table 6 were prepared in the manner described below. In Table 6, the gelling agent is carrageenan, the gelling aid is potassium chloride, the coloring agent is titanium oxide, and the plasticizer is D-sorbitol.

### <Preparation Method>

(1) Add the gelling aid (potassium chloride) to purified water at about 80° C. and dissolve. Further add the gelling agent (carrageenan) to the mixture, and dissolve with stirring. Add hydroxypropyl methylcellulose (HPMC) next with stirring, and disperse it in the mixture. Cool this solution to 50° C. while stirring until HPMC dissolves. Heat the solution to 55° C. with stirring, and then add the coloring agent (titanium oxide) and plasticizer (D-sorbitol) to obtain an aqueous capsule-preparing solution (dipping solution).

(2) Dip capsule-mold pins into the dipping solution thus obtained.

(3) Withdraw the pins from the dipping solution, and then allow the dipping solution adhering to the pins to gel at 35° C. or less.

(4) Dry the dipping solution adhering to the pins to form a capsule film. Then, remove the capsule film from the pins, and cut to a predetermined size.

(5) Heat-treat the pieces of capsule film at 70° C. to obtain the hard capsules of the invention.

TABLE 6

| | Examples | | | | | | | | | | |
|---|---|---|---|---|---|---|---|---|---|---|---|
| Composition | 3 | 4 | 5 | 6 | 7 | 8 | 9 | 10 | 11 | 12 | 13 |
| HPMC | 93.070 | 99.010 | 92.611 | 96.551 | 97.537 | 80.600 | 74.950 | 90.000 | 98.400 | 83.300 | 98.000 |
| Gelling agent | 0.372 | 0.396 | 0.926 | 0.966 | 0.975 | 2.200 | 10.000 | 10.000 | 1.100 | 1.200 | 1.500 |
| Gelling aid | 0.558 | 0.594 | 0.463 | 0.483 | 0.488 | 2.200 | 0.050 | 0 | 0.500 | 0.500 | 0.500 |
| Coloring | 6 | 0 | 6 | 2 | 0 | 10 | 10 | 0 | 0 | 10 | 0 |
| agent | | | | | | | | | | | |
| Plasticizer | 0 | 0 | 0 | 0 | 1 | 5 | 5 | 0 | 0 | 5 | 0 |
| Total | 100.0 | 100.0 | 100.0 | 100.0 | 100.0 | 100.0 | 100.0 | 100.0 | 100.0 | 100.0 | 100.0 |

All of these hard capsules had loss on drying of less than 6 wt % after they had been kept at 25° C. and at a relative humidity of 53% or less for 10 days. In addition, all of these capsules met at least one of the following requirements (a) to (e) as to the moisture absorption properties (%) at 25° C. and the following relative humidities.

(a) Moisture absorption properties of 9.2% or less at a relative humidity of 12%

(b) Moisture absorption properties of 9.5% or less at a relative humidity of 22%

(c) Moisture absorption properties of 9.7% or less at a relative humidity of 33%

(d) Moisture absorption properties of 10.9% or less at a relative humidity of 43%

(e) Moisture absorption properties of 11.1% or less at a relative humidity of 53%

### INDUSTRIAL APPLICABILITY

The hard capsule of the invention has a lower moisture content than not only gelatin capsules but also hitherto-known hard capsules produced by a gel cooling process using a water-soluble cellulose compound and a gelling agent as principal components (i.e., cold-gel hard capsules). Moreover, the hard capsule of the invention is imparted with favorable strength (impact resistance) as with hitherto-known cold-gel hard capsules even under low moisture conditions. The film of the hard capsule of the invention exhibits low hygroscopicity, and thus can be suitably used to contain substances that are likely to be affected by moisture. Furthermore, the hard capsule of the invention can be easily produced at low cost using a hitherto-known apparatus for producing cold-gel hard capsules by dip coating, while requiring no major investments in facilities nor special operations.

1. A hard capsule comprising a water-soluble cellulose compound, a gelling agent, and, if required, a gelling aid;
the hard capsule having loss on drying, after 10 days of storage at 25° C. and at a relative humidity of 53%, of less than 6% by weight.

2. The hard capsule according to claim 1, wherein hygroscopicity (%) of a capsule film under conditions of a temperature of 25° C. and each relative humidity value shown below satisfies at least one of the following conditions (1) to (5):
(1) 9.2% or less at a relative humidity of 12%;
(2) 9.5% or less at a relative humidity of 22%;
(3) 9.7% or less at a relative humidity of 33%;
(4) 10.9% or less at a relative humidity of 43%;
(5) 11.1% or less at a relative humidity of 53%.

3. The hard capsule according to claim 1, wherein the water-soluble cellulose compound is cellulose ether substituted with at least one group selected from C₁₋₆ alkyl groups and C₁₋₆ hydroxyalkyl groups.

4. The hard capsule according to claim 3, wherein the water-soluble cellulose compound is hydroxypropyl methylcellulose.

5. The hard capsule according to claim 1, wherein the gelling agent is at least one member selected from the group consisting of carrageenan, pectin, xanthan gum, locust bean gum, tamarind seed polysaccharide, curdlan, gelatin, fur selenium, agar, and gellan gum.

6. The hard capsule according to claim 5, wherein the gelling agent is carrageenan.

7. The hard capsule according to claim 1, wherein the water-soluble cellulose compound is hydroxypropyl methylcellulose, the gelling agent is carrageenan, and the gelling aid is potassium chloride.

8. The hard capsule according to claim 7, comprising 70 to 99.9% by weight of hydroxypropyl methylcellulose as the water-soluble cellulose compound, 0.05 to 2.2% by weight of carrageenan as the gelling agent, and 0.05 to 2.2% by weight of potassium chloride as the gelling aid.

9. The hard capsule according to claim 1 prepared by the following steps:
(1) a step of dipping a capsule-mold pin into a capsule-preparing solution comprising a water-soluble cellulose compound and a gelling agent, and, if required, a gelling aid;
(2) a step of drawing the capsule-mold pin out of the capsule-preparing solution, and gelling the solution adhering to an outside surface of the capsule-mold pin at a temperature of 35° C. or less;
(3) a step of drying the gelled capsule film formed covering the outside surface of the capsule-mold pin;
(4) a step of removing the dried capsule film from the capsule-mold pin, and
(5) a step of heating the gelled and/or dried capsule film to 50 to 150° C.; after step (2); before, after, or simultaneously with step (3); or after step (4).

10. The hard capsule according to claim 9, which is prepared by performing the heating step before, after, or simultaneously with step (3) of drying the gelled capsule film formed covering the outside surface of the capsule-mold pin.

11. A capsule product in which an ingredient is inserted into a hard capsule of claim 1.

12. The capsule product according to claim 11, in which the ingredient is a drug, food, or cosmetic material.

13. A method for producing a hard capsule comprising:
(1) dipping a capsule-mold pin into a capsule-preparing solution comprising a water-soluble cellulose compound and a gelling agent, and, if required, a gelling aid;
(2) drawing the capsule-mold pin out of the capsule-preparing solution, and gelling the solution adhering to an outside surface of the capsule-mold pin at a temperature of 35° C. or less;
(3) drying the gelled capsule film formed covering the outside surface of the capsule-mold pin;
(4) removing the dried capsule film from the capsule-mold pin; and
(5) heating the gelled and/or dried capsule film to 50 to 150° C.; after step (2); before, after, or simultaneously with step (3); or after step (4).

14. The method for producing a hard capsule according to claim 13, comprising the heating step before, after, or simultaneously with step (3) of drying the gelled capsule film formed covering the outside surface of the capsule-mold pin.

15. The method for producing a hard capsule according to claim 13, the hard capsule having loss on drying, after 10 days of storage at 25° C. and at a relative humidity of 53%, of less than 6% by weight.

16. A method for reducing a moisture content and hygroscopicity of a hard capsule comprising as a main component a water-soluble cellulose compound, a gelling agent, and, if required, a gelling aid, the method comprising:
(a) dipping a capsule-mold pin into a capsule-preparing solution comprising a water-soluble cellulose compound and a gelling agent, and, if required, a gelling aid;
(b) drawing the capsule-mold pin out of the capsule-preparing solution, and gelling the solution adhering to an outside surface of the capsule-mold pin at a temperature of 35° C. or less;
(c) drying the gelled capsule film formed covering the outside surface of the capsule-mold pin; and
(d) removing the dried capsule film from the capsule-mold pin,
wherein the gelled and/or dried capsule film is heated to 50 to 150° C.; after step (b); before, after, or simultaneously with step (c); or after step (d).
